(19) United States
(12) Patent Application Publication
(10) Pub. No.: US 2024/0151678 A1
(43) Pub. Date: May 9, 2024

(54) OSCILLATING REACTION DEVICES, SYSTEMS, AND METHODS FOR SOLVING ISING PROBLEMS

(71) Applicant: INTERNATIONAL BUSINESS MACHINES CORPORATION, ARMONK, NY (US)

(72) Inventors: Armin Knoll, Adliswil (CH); Heiko Wolf, Pfaeffikon (CH); Philippe Marc Nicollier, Sion (CH); Daniel Widmer, Langnau am Albis (CH); Jacqueline Figueiredo da Silva, Zurich (CH)

(21) Appl. No.: 18/052,989

(22) Filed: Nov. 7, 2022

(51) Int. Cl. G01N 27/28; G01N 27/416; G06N 3/12; G16B 50/30

(52) U.S. Cl. CPC G01N 27/286; G01N 27/416; G06N 3/123; G16B 50/30

(57) ABSTRACT

The invention is notably directed to an oscillating reaction device. The device basically includes a structured layer of a matrix material and structural elements. Cavities are defined (for example, as blind holes) in the structured layer to allow the cavities to be filled with an oscillating reaction liquid containing nonpolar molecules of a given molecular species, with a view to triggering an oscillating reaction. Centers of the cavities are arranged according to a 2D lattice, whereby pairs of nearest-neighbor cavities of said cavities are, each, separated by a portion of the matrix material. This portion of material is selectively permeable to said nonpolar molecules. This, in operation, yields an inhibitory chemical coupling of the filled cavities of said pairs of nearest-neighbor cavities, where the inhibitory chemical coupling is mediated by said nonpolar molecules. Also, the structural elements connect the cavities to yield a positive chemical coupling of the filled cavities.

### STATEMENT REGARDING FEDERALLY SPONSORED RESEARCH OR DEVELOPMENT

The project leading to this application has received funding from the European Union's Horizon 2020 research and innovation program under Marie Sklodowska-Curie, grant number 812868.

### BACKGROUND

The invention generally concerns techniques (devices, systems, and methods) related to oscillating reactions. In particular, it is directed to a device including cavities meant to be filled with an oscillating reaction liquid, to trigger an oscillating reaction, where the device is configured to allow both an inhibitory chemical coupling and a positive chemical coupling of the filled cavities. The device can notably be used as a molecular Ising solver.

Molecular computing is a branch of computing that uses DNA (Deoxyribonucleic acid), biochemistry, and molecular biology hardware, instead of traditional (silicon-based) electronic computing. There were early attempts to use simpler molecules, mostly in oscillating chemical reactions such as the Belousov Zhabotinsky (BZ) reaction. However, the setups proposed so far are usually not scalable and thus not suited for achieving competitive performance.

### SUMMARY

According to a first aspect, the present invention is embodied as an oscillating reaction device. The device basically includes a structured layer of a matrix material and structural elements. Cavities are defined (for example, as blind holes) in the structured layer to allow the cavities to be filled with an oscillating reaction liquid containing nonpolar molecules of a given molecular species, with a view to triggering an oscillating reaction. Centers of the cavities are arranged according to a 2D (two dimensional) lattice, whereby pairs of nearest-neighbor cavities of said cavities are, each, separated by a portion of the matrix material. This portion of material is selectively permeable to said nonpolar molecules. This, in operation, yields an inhibitory chemical coupling of the filled cavities of said pairs of nearest-neighbor cavities, where the inhibitory chemical coupling is mediated by said nonpolar molecules. In addition, the structural elements connect the cavities to yield a positive chemical coupling of the filled cavities.

The structural elements may for instance define a flow path between the cavities. They may also include electrical connectors, each connecting interiors of cavities of a respective pair of nearest-neighbor cavities.

In embodiments, the device further includes electrical contacts defined to allow electrochemical measurements to be performed from within the cavities, in operation. The device may for instance be configured to allow pulsed signals to be applied to the cavities to force said oscillating reaction in accordance with an external clock, in operation.

Another aspect of the invention concerns an oscillating reaction system, which includes an oscillating reaction device as described above, as well as a readout unit. The readout unit is configured to allow electrochemical or optical measurements to be performed from within the cavities of each pair of nearest-neighbor cavities. The readout unit may for instance be connected to the cavities via electrical contacts, to allow electrochemical measurements. In variants, optical readouts are performed.

In preferred embodiments, the system further includes a signal generator, which is coupled to the oscillating reaction device and configured to apply excitatory signal pulses to the cavities. The system further includes a control unit, which is connected to the signal generator to transmit clock signals to the signal generator at a given frequency, for the signal generator to apply the signal pulses in accordance with the transmitted clock signals and thereby force the oscillating reaction across the cavities at said oscillation frequency.

Preferably, the system further comprises a computer interfaced with each of the control unit and the readout unit. The computer may for instance store parameters of a quadratic unconstrained binary optimization (QUBO) problem, or an Ising problem. The computer may further be configured to generate and transmit signals to the control unit and trigger said measurements in accordance with said parameters.

According to another aspect, the invention is embodied as a method of operating an oscillating reaction device or a system such as described above. According to the method, an oscillating reaction liquid is filled into the cavities. The oscillating reaction liquid contains nonpolar molecules of the given molecular species. This triggers an oscillating reaction that is subject to both a positive chemical coupling and an inhibitory chemical coupling of the filled cavities of the pairs of nearest-neighbor cavities. The positive chemical coupling results from the structural elements. The inhibitory chemical coupling is mediated by the nonpolar molecules; it is governed by a dimension of the portion of the matrix material separating the coupled cavities. The method further comprises performing electrochemical or optical measurements from within the cavities, for example, with a view to solving a QUBO problem or an Ising problem.

In preferred embodiments, the method further comprises applying signal pulses to the cavities according to an oscillation frequency to force the oscillating reaction across the cavities at said oscillation frequency.

According to an aspect of the present invention, an oscillating reaction device includes: (i) a structured layer of a matrix material; and (ii) a plurality of structural elements. The plurality of cavities are defined in the structured layer, with the cavities having a size and shape such that the cavities can be filled with an oscillating reaction liquid that includes nonpolar molecules of a given molecular species to trigger an oscillating reaction. Respective central axes of the cavities are arranged according to a 2D lattice sized and shaped such that each pair of nearest-neighbor cavities of the plurality of cavities is separated by a portion of the matrix material. The portions of matrix material separating each pair of cavities is selectively permeable to the nonpolar molecules, to yield an inhibitory chemical coupling of filled cavities of each pair of nearest neighbor cavities so that the inhibitory chemical coupling is mediated by the nonpolar molecules in operation. The structural elements of the plurality of structural elements connect the cavities to yield a positive chemical coupling of the filled cavities, in operation.

According to a further aspect of the present invention, a method of operating an oscillating reaction device includes the following operations: (i) providing a reaction device comprising a structured layer of a matrix material, in which cavities are defined and centers of the cavities are arranged according to a 2D lattice, whereby pairs of nearest-neighbor cavities of the cavities are, each, separated by a portion of the matrix material, the portion being selectively permeable to nonpolar molecules of a given molecular species and structural elements connecting the cavities; (ii) filling an oscillating reaction liquid into the cavities, the oscillating reaction liquid containing nonpolar molecules of the given molecular species, to trigger an oscillating reaction that is subject to a positive chemical coupling of filled cavities of the pairs, the positive chemical coupling resulting from the structural elements and an inhibitory chemical coupling of the filled cavities of the pairs, the inhibitory chemical coupling mediated by the nonpolar molecules and governed by a dimension of the portion of the matrix material separating the cavities of each of the pairs; and (iii) performing electrochemical or optical measurements from within the cavities.

According to a further aspect of the present invention, a device includes: (i) a structured layer of a matrix material; (ii) a plurality of structural elements; and (iii) an oscillating reaction liquid that includes nonpolar molecules of a given molecular species suitable for being subject to an oscillating reaction. A plurality of cavities are defined in the structured layer. The cavities of the plurality of cavities are filled with the oscillating reaction fluid. Respective central axes of the cavities are arranged according to a two dimensional lattice sized and shaped such that each pair of nearest-neighbor cavities of the plurality of cavities is separated by a portion of the matrix material. The portions of matrix material separating each pair of cavities is selectively permeable to the nonpolar molecules, to yield an inhibitory chemical coupling of filled cavities of each pair of nearest neighbor cavities so that the inhibitory chemical coupling is mediated by the nonpolar molecules during the oscillating reaction. The structural elements of the plurality of structural elements connect the cavities to yield a positive chemical coupling of the filled cavities during the oscillating reaction.

### BRIEF DESCRIPTION OF THE DRAWINGS

These and other objects, features and advantages of the present invention will become apparent from the following detailed description of illustrative embodiments thereof, which is to be read in connection with the accompanying drawings. The illustrations are for clarity in facilitating one skilled in the art in understanding the invention in conjunction with the detailed description. In the drawings:

FIGS. 1A-1C show wireframes of a device according to embodiments. FIGS. 1A, 1B, and 1C, respectively show a side view, a top view, and a 3D view of the device.

FIG. 2 depicts a device as in FIG. 1A, to which global light pulses are applied, thanks to a light source that is synchronized and modulated according to an external clock, whereby pulsed optical signals are applied to the cavities of the device to force an oscillating reaction in the cavities to synchronize with the external clock, as in embodiments;

FIG. 3 shows a cross sectional view of a device as in FIG. 1A and illustrates how an inhibitory chemical coupling of the filled cavities can be mediated by nonpolar molecules (here Br2) through a lateral portion of a matrix material of the device, as in embodiments;

FIG. 4 is a variant to the device shown in FIG. 1A, in which the electrical connectors that connect interiors of the cavities further include a programmable resistive element, as in embodiments;

FIG. 5 is a top view of a network of cavities arranged according to a 2D hexagonal lattice, where the cavities are connected by phase change memory elements, which can be programmed by applying light through a transparent layer of the device, as in embodiments;

FIG. 6 is a top view of a subset of cavities of a reaction device, where at least some pairs of nearest-neighbor cavities are connected by metal wires of distinct electrical resistances, as involved in embodiments;

FIG. 7 is a high-level wiring diagram of an oscillating reaction system according to embodiments;

FIG. 8 is a schematic representation of an oscillating reaction system according to embodiments; and

FIG. 9 is a flowchart illustrating high-level steps of a method of operating a system such as depicted in FIG. 8, according to embodiments.

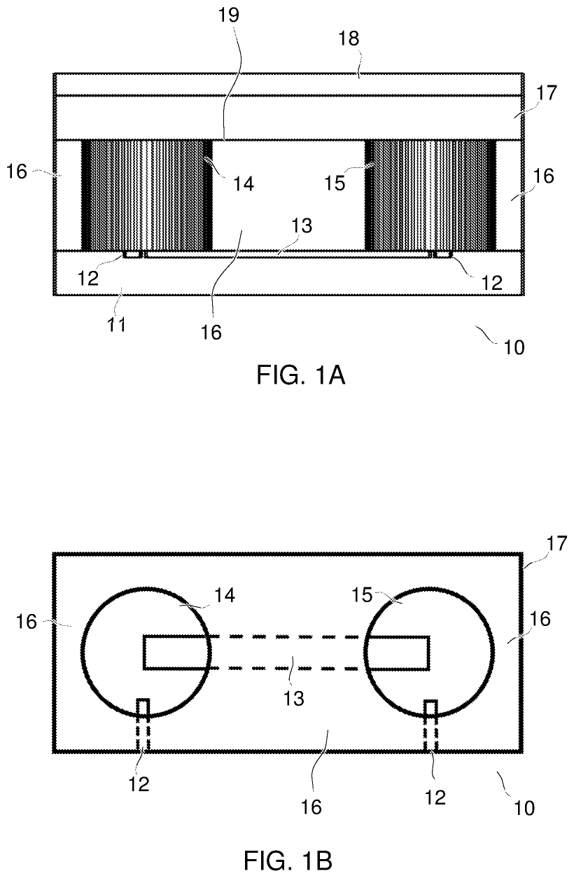

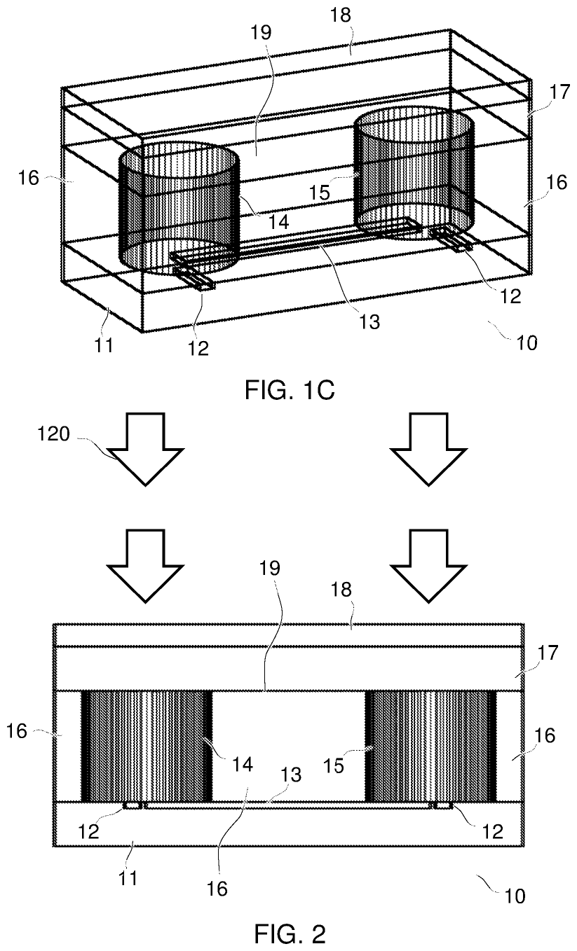

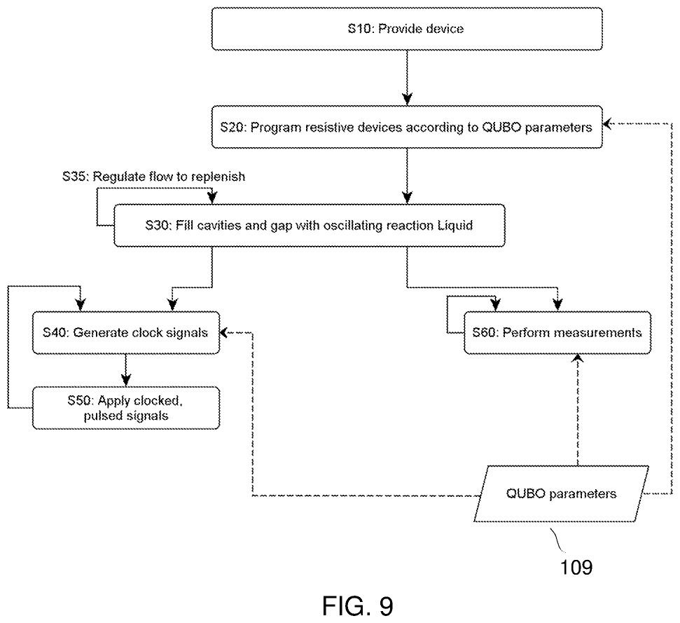

The accompanying drawings show simplified representations of devices, systems, or parts thereof, as involved in embodiments. Technical features depicted in the drawings are not necessarily to scale. FIGS. 1A-4 only depict a pair of closest cavities, for conciseness. Similarly, FIG. 6 shows a selection of seven cavities; reaction devices according to embodiments may contain many more cavities. Similar or functionally similar elements in the figures have been allocated the same numeral references, unless otherwise indicated. All references Sn refer to methods steps of the flowcharts of FIG. 9, while numeral references pertain to devices, components, and systems involved in embodiments of the present invention.

Devices, systems and methods embodying the present invention will now be described, by way of non-limiting examples.

### DETAILED DESCRIPTION OF EMBODIMENTS OF THE INVENTION

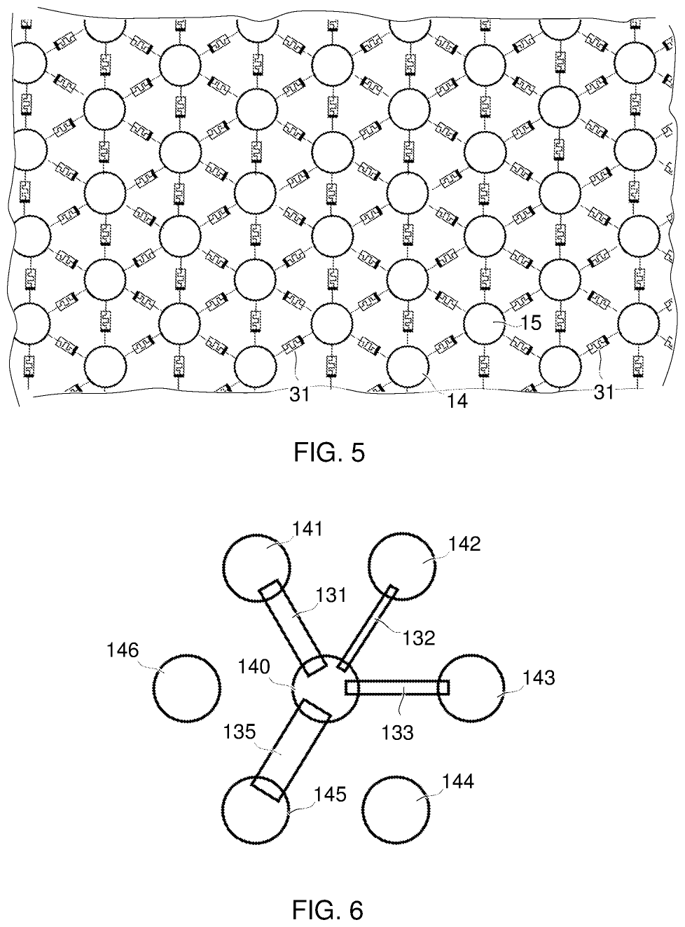

A first aspect of the invention is now described in detail in reference to FIGS. 1A-6. This aspect concerns an oscillating reaction device 10, 10a, which is designed to enables molecular computing based on organic and/or inorganic chemistry. Reference 10 concerns a first embodiment of the device, shown in FIGS. 1A-3, while reference 10a pertains to a second embodiment, involving programmable resistances. Again, FIGS. 1A-4 are partial depictions, involving a single pair of cavities. In practice, however, the oscillating reaction device may include a larger number of cavities, as illustrated in FIG. 5.

Oscillating reaction device 10, 10a essentially includes a structured layer 16 of a matrix material, as well as structural elements, such as electrical connectors 13 and a lid 18. Lid 18 may, together with top surface 19 of layer 16, notably define a gap g. In variants, the device may include structural elements defining a trench. In addition, device 10, 10a may include electrical contacts 12 and programmable resistive elements 31, as in embodiments discussed later in detail.

Cavities 14, 15 are defined in structured layer 16. Such cavities are meant to be filled with an oscillating reaction liquid, which notably contains nonpolar molecules of a given molecular species, such as Br2 molecules. Such molecules are also referred to as “mediating molecules” in this document, for reasons explained below. The aim of filling the cavities with the reaction liquid is to trigger an oscillating reaction, such as a Belousov Zhabotinsky (BZ) reaction. The oscillating reaction liquid contains various other species, as known per se.

Cavities 14, 15 are preferably defined as blind holes in structured layer 16. That is, the cavities are open on top surface 19 of structured layer 16, that is, on the top side of layer 16 in FIGS. 1A-4. As a result, cavities 14, 15 of each pair are laterally separated by a portion of matrix material 16. This portion extends along a direction that is parallel to top surface 19, over a distance t. Now, this distance can be chosen to be less than or equal to an average diffusion distance of the mediating molecules through the matrix material 16 over a period of the oscillating reaction, such that these molecules can appreciably diffuse through structured layer 16 from one cavity to the next during such a period. That is, the diffusion distance can be defined as the average distance over which the mediating molecules diffuse over a certain time. In the present context, the relevant time scale is the period of the oscillating reaction. Thus, it should preferably be on the order of this diffusion distance, or smaller. The diffusion of the mediating molecules through the matrix material 16 results in an inhibitory coupling of the filled cavities, in operation.

As for instance illustrated in FIG. 5, the centers of the cavities can be arranged according to a 2D lattice. Because cavities 14, 15 are defined in the matrix material of layer 16, the 2D lattice arrangement results in that each pair of nearest-neighbor cavities are separated by essentially a same portion of matrix material 16. The dimension of this portion is determined by the 2D lattice step. As explained above, this portion can be dimensioned so as for the corresponding portion of matrix material to be selectively permeable to the mediating molecules of the oscillating reaction liquid. In practice, the separation distance t is preferably between 1 and 10 μm. Such a configuration gives rise to an inhibitory chemical coupling of the filled cavities of the pairs of nearest-neighbor cavities, where the inhibitory chemical coupling is mediated by the nonpolar molecules, that is, the mediating molecules, in operation. The dimensions of the portions of materials separating the nth neighbors (n>2) gradually increases, becoming less and less permeable to the nonpolar molecules. In practice, permeation only occurs between first-neighbor cavities, whereby the inhibitory chemical coupling is determined by the pairs of first-neighbor cavities only.

Conversely, structural elements 13, 18, 19 connect cavities 14, 15 to yield a positive chemical coupling of the filled cavities, in operation. The positive chemical coupling concerns pairs of first-neighbor cavities and may further extend to farther cavities, this depending on how the structural elements connect them. Examples are discussed later in detail.

Thus, two effects compete, namely the positive chemical coupling and the inhibitory chemical coupling, which govern the oscillating reaction taking place in the device. Overall, the effective coupling is a superposition of the individual coupling mechanisms. Accordingly, a continuous overall coupling strength can be achieved (at least in a certain range), ranging from positive to negative values, something that can be exploited to solve quadratic unconstrained binary optimization (QUBO) problems or Ising problems, as described in detail in reference to the present method, that is, final aspect of the invention.

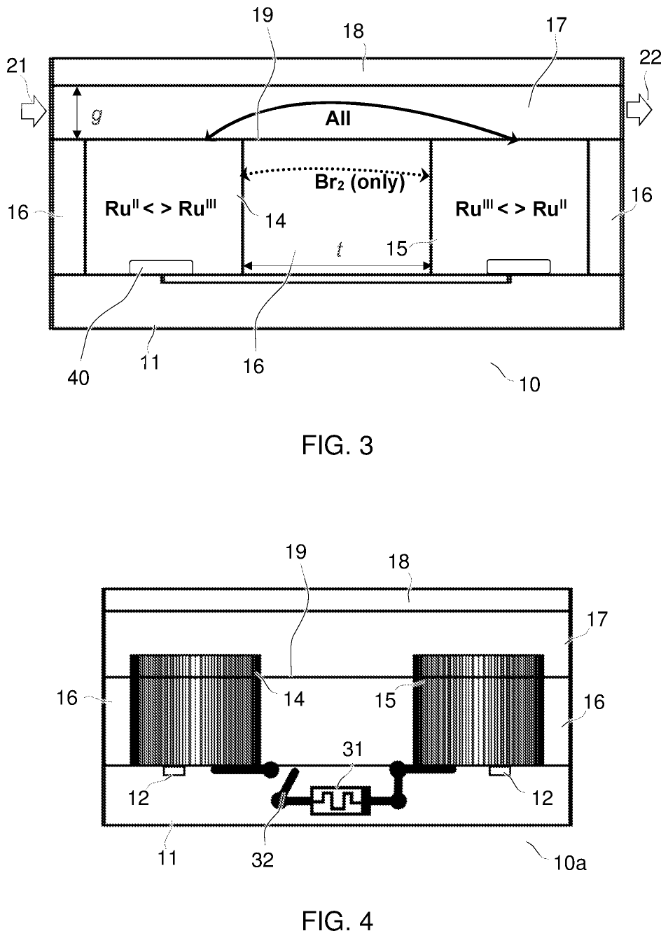

Comments are in order. As a whole, device 10, 10a must be designed in such a manner that the oscillating reaction liquid reach and fill the cavities, with a view to triggering the oscillating reaction, in operation. In that respect, the device is preferably designed to define a flow path 17 (for example, thanks to a gap g provided above matrix material 16), which connects cavities 14, 15, so as to allow the reaction liquid to effectively reach and fill the cavities, in operation. In addition, the device may typically include a liquid inlet 21 and a liquid outlet 22 (see FIGS. 3 and 8), which are arranged to allow cavities 14, 15 to be replenished with the oscillating reaction liquid, if necessary. That is, fresh liquid can be injected via the inlet while excess liquid can be discharged from the outlet, possibly in a continuous manner. If necessary, inlet 21, outlet 22, and flow path 17, can be processed to have wetting surfaces, with respect to the oscillating reaction liquid.

The present devices rely on lithographically-obtained cavities 14, 15 (that is, cells), which define chemical volumes that can notably be used for computations. For example, Ising-like interactions can be implemented through the geometry and arrangement of such chemical volumes. The chemical couplings result from the relative arrangement of the cavities, their separations, and structural elements 13, 18, g that couple the cavities.

In particular, the way the cavities are defined in the matrix material allows an inhibitory coupling to be achieved between the filled cavities, owing the limited thickness of the wall portion of matrix material 16 separating the cavities, laterally. The matrix material 16 ensures an inhibitory coupling through molecular diffusion through the inter-cavity walls, the coupling strength of which can be adjusted by the wall thickness between the cavities. The inhibitory coupling is a negative coupling in the sense of the underlying chemical reaction principle, which leads to out-of-phase oscillations in the oscillating reaction.

The portions of matrix material separating adjacent cavities can be regarded as semipermeable material portions, which are selectively permeable to small molecules (but not to larger particles). In the present context, the permeant of interest includes nonpolar molecules, which mediate the inhibitory chemical coupling, by diffusion through the matrix material. Thus, the extent to which the matrix material is permissive to diffusion by the permeant impacts the inhibitory coupling.

Various matrix materials 16 can be contemplated, starting with nonpolar materials such as polydimethylsiloxane (PDMS) or a SU-8 photoresist. Other photoresist materials can be used too. A nonpolar material is a material having no net dipole. In the case of polymers, a non-polar material means that the polymer should have no polar groups, in principle. However, what is fundamentally needed, in the present context, is a material having a sufficient permeability to small nonpolar molecules such as Br2. Some polymers have a sufficient permeability to small nonpolar molecules, despite the presence of a polar group.

For instance, the matrix materials 16 may possibly include polymethyl methacrylate (PMMA). Such a material is “polar”, inasmuch as it has a polar side group. However, such a material should, in principle, allow permeation of small nonpolar molecules over small distances. Moreover, porous materials can be used, such as porous silicon oxide, as well as ceramics. As noted above, top surface 19 of this material may possibly be processed, to ensure a wetting flow path. This surface can notably be a hydrophilic surface, although the bulk of material 16 can be hydrophobic.

The permeant is formed by nonpolar molecules of a given molecular species, which are adapted to mediate an inhibitory chemical coupling through the chosen material, between the filled cavities. In the present context, the permeant is typically a small diatomic molecule, such as Br2, which lends itself well to oscillating reactions as contemplated herein.

Conversely, a positive coupling is enabled by structural elements 13, 18, g. In particular, a gap can be defined on top of top surface 19. In addition, or in variant, electrical conductors such as lithographically defined wires 13 can be patterned in or on a basis layer 11, below matrix material 16, to ensure a positive coupling of defined strength. In variant, the wires can also be defined on or in lid 18 delimiting gap g. Wires 13 can notably be made of a (noble) metal or a highly-doped semiconductor. Another possibility is to rely on programmable resistive elements 31. The positive coupling is an excitatory coupling, which leads to synchronization, that is, in-phase oscillations in an oscillating reaction such as the BZ reaction.

The positive coupling competes with the inhibitory coupling. Still, the strength of such couplings can be adjusted, by design, and/or by adjusting resistances of wires 13 or by programming resistances of programmable resistive elements 31 linking the first-neighbor cavities. So, it is possible to adjust the effective coupling between the cavities.

A key advantage of present devices 10, 10a is the possibility to achieve a defined coupling strength between cavities of lithographically-defined geometry. In particular, the coupling strength between first-neighbor cavities can be adjusted from positive to negative. Thus, complex Hamiltonians can be implemented, in accordance with QUBO problems, which are a class of problems including Ising problems.

Accordingly, the proposed approach makes it possible to achieve a molecular-based, information processing device or system, the elements (that is, cavities, separation distances, gaps, connectors) of which can be scaled to very small dimensions (that is, nano to micrometer scale), with high performance at low energy consumption. The proposed architecture is scalable, and the underlying concept allows for a very high parallelism in the computations, thus providing a non-conventional, albeit efficient, way for solving complex computational problems such as Ising problems.

All this is now described in detail, in reference to particular embodiments of the invention. To start with, the chemical reaction oscillations may advantageously be forced by applying electrical signals in accordance with an external clock. That is, device 10, 10a can be configured to allow pulsed signals to be applied, either optically or electrically via electrical contacts 12, to cavities 14, 15, to force the oscillating reaction in the cavities in accordance with the external clock. The device may notably form part of system 1 including adequate electromagnetic or electric pulsing means, as discussed later.

Note, however, that the reaction oscillations are an intrinsic feature of the chemistry at issue; they do not require external triggers. The signals applied are only needed to impose an external clock, that is, to force the coupling in accordance with this external clock, something that can be exploited to solve Hamiltonian-based problems such as Ising problems.

Where electromagnetic pulsing means are used (as assumed in FIGS. 2 and 3), device must allow the pulsed light 120 to reach the cavities, globally. That is, a same light source illuminate all cavities at the same time. To that aim, the device may include a top and/or bottom layer that is permissive to light (in the necessary spectral range). For example, lid 18 in device 10 of FIG. 2 is assumed to be permissive to the pulsed light, such that optical pulses can reach the liquid in the cavities, in operation.

In variants, use can be made of electrical contacts 12 to apply pulsed electric signals. Interestingly, the same contacts 12 (or similarly arranged contact) can also be used to perform electrochemical measurements, via electrical conductors (not shown) contacting the electrical contacts 12. Thus, present devices 10, 10a preferably include electrical contacts 12, which can notably be arranged, at least partly in or on a basis layer 11 of the device, as illustrated in FIGS. 1A-4. This way, electrical contacts 12 are in contact with the liquid filling the cavities, in operation. Such electrical contacts 12 allow electrochemical measurements to be performed from within cavities 14, 15. In variants, contacts 12 are formed as through-silicon vias (TSVs). That is, through holes can be processed in layer 11, and filled with metal. The resulting array of TSVs makes it possible to electrically connect to all cavities in the device.

In variants, the readouts can also be performed optically, for example, using fluorescence. A suitable fluorescent marker is tris-(2,2?-bipyridyl)-ruthenium(II)2+ complex, as assumed in FIG. 3. Note, this marker changes according to the redox state and, thus, with the electrochemical potential.

As noted earlier, structural elements 18, g can notably be designed to define a flow path between pairs of adjacent cavities 14, 15 or, even, between multiple cavities. That is, a same flow path can connect multiple cavities, possibly all cavities of device 10, 10a. When filled by the oscillating reaction liquid, this flow path forms a diffusion flow path, through which various species (including the mediating molecules) can diffuse, that is, from one cavity to another. Unlike the diffusion occurring through the lateral portions of matrix material 16, the diffusion process enabled by filled flow path 17 will at least contribute to the positive chemical coupling of the filled cavities. Note, the flow path allows diffusion between adjacent cavities (that is, the first-neighbor cavities). However, diffusion may also occur between farther cavities.

In particular, the structural elements may be arranged to define a gap g, which itself defines the flow path, as assumed in FIGS. 1A-4. In the accompanying drawings, the gap g is defined by top surface 19 of the matrix material 16 and lid 18, which extends over top surface 19, at a distance thereof. Liquid inlet 21 and liquid outlet 22 can be set in fluid communication with the gap g, which can thus be filled by the oscillating reaction liquid to form the diffusion flow path, in operation. The dimension of gap g (as measured perpendicularly to top surface 19) is typically between 0.001 and 1 μm, though it is preferably between 10 and 500 nm, and more preferably between 10 and 100 nm.

Gap g can be regarded as a channel serving multiple cavities. In variants, however, the device may include additional microstructures (for example, forming grooves or conduits defining one or more channels), without necessarily resulting in a gap on top of top surface 19. Such microstructures may notably form one or more trenches.

Where a gap g is used to connect the cavities, diffusion occurs between distant cavities, too. However, where trenches are used to connect the cavities, diffusion transports molecules from one cavity to the next. In each case, the small quantity of molecules passing from one cavity to a distant cavity (for example, a second- or third-neighbor cavity) should, in principle, change the oscillating behavior of the distant cavity. However, this cavity already contains a much larger number of the same molecules. In that sense, the effect of the diffusion is effectively measurable between first-neighbor cavities only. Similarly, diffusion through the walls is appreciable between first-neighbor cavities only.

The above examples involve gaps or trenches. In each case, one or more channels are formed, which define a flow path. And in each of these cases, a liquid inlet 21 and a liquid outlet 22 can be set in fluid communication with this flow path, to allow cavities 14, 15 to be replenished with the oscillating reaction liquid, if necessary. That being said, it may not always be necessary to replenish the cavities because the chemical reaction can typically last for approximately 100 to 150 reaction cycles, even without refilling the device. Now, this may already be sufficient to arrive at a solution of the problem to be solved. So, it is not always needed to replenish the device, such that the liquid inlet and the liquid outlet are optional. For example, the cavities may possibly be filled from top and device 10, 10a may possibly be closed (with lid 19) right after filling the cavities.

In embodiments, the structural elements further include electrical connectors 13, as illustrated in FIGS. 1A-3. Each electrical connector 13 connects interiors of cavities 14, 15 of a respective pair of nearest-neighbor cavities. The electrical connectors can be mere electrical conductors, such as wires (for example, patterned conductors, as assumed in FIGS. 1A-3). For example, a lithographically defined wire of a noble metal (for example, Pt, Au) can be patterned on top of a basis layer 11, so as to be exposed at the bottom of the cavities. Thus, the wire comes in direct contact with the reaction liquid filling adjacent cavities, thereby connecting the two corresponding chemical volumes. This leads to a positive coupling, the strength of which is proportional to the exposed area of the wire. In more sophisticated variants, the electrical connectors may include conductive tracks, forming portions of electric circuits 31, 32, which may possibly include programmable resistances 31 and switches 32, as schematically depicted in FIG. 4.

Like the diffusion flow paths described above, such electrical connectors 13, 31, 32 contribute to the positive coupling. Still, embodiments may solely involve electrical connectors 13, 31, 32 or diffusion flow paths g. In the latter case, the connection is ensured by diffusion through a fluidic connection, while it is an electrical connection in the former case. Each type of connection allows an electrochemical connection, and thus a chemical coupling, to be achieved. Still, preferred embodiments rely on both electrical conductors 13, 31, 32 and a diffusion flow path, because this ensures additional leeway to tune the chemical coupling. In each case, some structural elements 13, 18, g connect pairs of cavities 14, 15 to allow a positive chemical coupling of the cavities to be achieved.

As noted above, electrical connectors 13, 31, 32 can be at least partly arranged in and/or on the basis layer 11, so as to connect interiors of the cavities of each pair of closest cavities. Connectors 13 can for instance be patterned on top of layer 11 or within a superficial thickness of this layer. So do contacts 12. In the examples of FIGS. 1A, 1B, 1C, 2, and 3, both electrical contacts 12 and connectors 13 are patterned within a superficial thickness of layer 11, so as to be flush with top surface of layer 11. Note, device 10, 10a may possibly include more than one basis layers. Such layers (not shown) extend parallel to structured layer 16, opposite to top side 19. That is, one or more additional layers may be provided below layer 11 shown in FIGS. 1A-4, for example, to ensure a sufficient mechanical resistance. Plus, one or more of such additional layers may accommodate electrical circuits or portions of such circuits, for example, be it to adequately connected to electrical contacts 12 and/or program resistive elements 31, if any.

FIG. 6 is a partial, top view of a device 10, 10a, showing a given cavity 140, surrounded by closest cavities 141, 142, 143, 144, 145, 146. As in FIG. 5, the cavities are distributed according to a 2D hexagonal lattice in this example. Central cavity 140 and its first-neighbor cavities 141-146 make up six first-neighbor pairs, some of which are connected by an electrical connector 131, 132, 133, 135. In this example, electrical connectors 131, 132, 133, 135 consist of noble metal wires 131, 132, 133, 135, which are patterned on a lower layer (not shown), so as to be exposed to interiors of connected cavities 140, 141, 142, 143, 145. Again, the noble metal wires can be lithographically defined on the basis layer that is directly underneath the cavities, or within a superficial thickness of this layer, so as to be level with the top surface of the basis layer. The length 1 of such wires must be larger than the thickness t of the portion of matrix material separating the first-neighbor cavities, to be able to contact the liquid. Conversely, they can be smaller than t+2×d/2, where d denotes the average diameter (in-plane) of the cavities.

Wires 131, 132, 133, 135 have distinct electrical resistances in the example of FIG. 6. Such resistances are chosen in accordance with the problem to solve. An easy way to achieve distinct electrical resistances is to pattern metal wires 131, 132, 133, 135 of distinct widths. The width of the wires determines the area coming in contact with the liquid, which in turn determines the resistance of the wires to the liquid. The width of the metal wires will likely be less than 10 μm in practice. The wire width is typically between 10 nm and 0.1 μm. However, the (out-of-plane) thickness of all wires can be the same, should this ease the lithographic fabrication process.

For example, device 10 may include a matrix layer 16 of a nonpolar material 16, such as PDMS or SU-8. The cavities can be processed in this layer 16 and distributed according to a 2D hexagonal lattice, so as to be open on top of matrix material layer 16. A gap g is ensured on top of this layer 16, which forms a diffusion flow path. Gap g is further delimited by a glass lid 18, which may rest on top of posts or other microstructures (not shown). Electrical contacts 12 and connectors 13 (noble metal wires) can be patterned on top of basis layer 11, which can be made of glass, silicon, or be a SiOx substrate, for example. The cavities may further include respective pieces of a catalyst material 40 (for example, a gel patch, a monolayer, or particles), where catalyst material 40 is preferably immobilized at the bottom of a respective cavity (as assumed in FIG. 3). Typically, the reaction liquid oscillates only in the diffusion distance of the catalyst; chemicals are much slower oxidizing outside the oscillating volume. Replenishment of the reaction liquid is ensured by liquid inlets and outlets in fluid communication with the gap.

The above example assume static resistances of the wires, which are determined by the wire dimensions. As evoked earlier, more sophisticated approaches can be contemplated, which rely on programmable resistive elements. For instance, the oscillating reaction device 10a shown in FIG. 4 has electrical connectors 31, 32 that include a programmable resistive element 31. That is, each pair of first-neighbor cavities is connected via a programmable resistive element 31, as depicted in FIG. 5. In addition, a switch 32 can be inserted in the electrical path connecting adjacent cavities, in order to activate the corresponding path, if necessary. Several types of permissive devices may be used, such as phase change memory (PCM) cells, resistive random-access memory (RRAM), static random-access memory (SRAM) cells, or electro-chemical random-access memory (ECRAM) cells. In other variants, flash cells may be used too.

Device 10a may further include a programming circuit (not shown) to program resistive elements 31 and change their resistive states. This circuit may for instance be provided in a layer underneath basis layer 11. The programming circuit may also form part of an external component. An easier option, however, is to rely on phase change memory elements 31, which can more easily be programmed by applying light thereto, through a layer permissive to light, as assumed in FIG. 5. Note, resistive elements 31 may advantageously enable several resistive states. For example, the actual resistive state of the phase change memory elements 31 may be determined by the duration of the applied optical signal, as known per se.

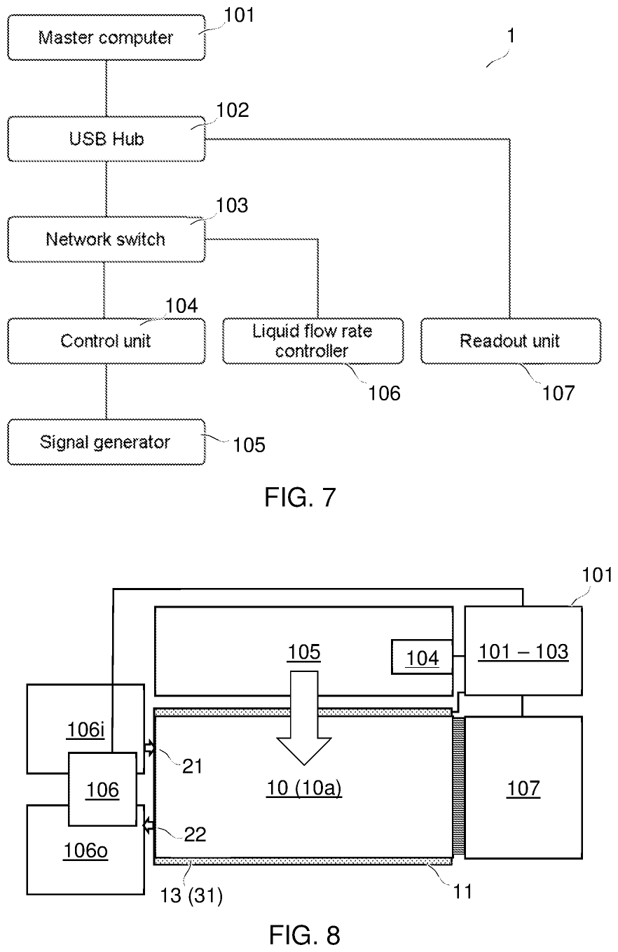

Another aspect of the invention is now described in reference to FIGS. 7 and 8. This aspect concerns an oscillating reaction system 1. System 1 primarily relies on an oscillating reaction device 10, 10a such as described above in reference to FIGS. 1A-6. In addition, system 1 includes a readout unit 107, which is connected to cavities 14, 15, to allow optical or electrochemical measurements to be performed from within the cavities, in operation. That is, readout unit 107 may be optically connected to the cavities, to allow optical readouts, as noted earlier. In variants, the readout unit is connected to the cavities via electrical contacts 12, to allow electrochemical measurements to be performed via contacts 12.

In embodiments, system 1 further includes a signal generator 105. Signal generator 105 is coupled to oscillating reaction device 10, 10a and configured to apply excitatory signal pulses (either optical or electrical signals) to cavities 14, 15. Moreover, a control unit 108 can be connected to signal generator 105, to transmit clock signals to the signal generator. The clock signals may be transmitted at a given frequency or somehow encode this frequency. In turn, signal generator 105 applies the signal pulses in accordance with the transmitted clock signals, that is, in accordance with the given frequency. This, in operation, causes to force the oscillating reaction across cavities 14, 15 of device 10, 10a at the given frequency. That is, the oscillation frequency corresponds to the given frequency.

As explained earlier, device 10, 10a may possibly include electrical contacts 12 contacting each of cavities 14, 15. Signal generator 105 may thus be electrically connected to such electrical contacts 12 to apply the signal pulses as electrical signal pulses. In variants, the signals are applied as optical pulses. Similarly, the measurements can be done optically instead of being performed via contacts 12, such that contacts 12 are optional.

System 1 may include additional components. For example, system 1 may include a computer 101, which is interfaced with control unit 108 and readout unit 107, to automate the operation of such units 107, 108 in accordance with parameters stored in the computer. In particular, computer 101 may store parameters 109 of a QUBO problem or an Ising problem. Computer 101 may notably be configured to generate and transmit signals to both readout unit 107 and control unit 108, with a view to triggering (optical or electrochemical) measurements and pulsed signals, in accordance with such parameters.

In variants, or in addition, signal generator 105 and readout unit 107 may possibly be connected in a feedback loop circuit. This circuit may be designed to ensure that signal generator 105 applies the excitatory signal pulses based on signals as read out by readout unit 107. The signals read by the readout unit encompass the optical or electrochemical measurements. Depending on the signals measured across all cavities, a weighted signal can be computed for each cavity, which signal corresponds to the coupling strength in the desired Hamiltonian. This signal is applied to a respective cavity to electrochemically influence its oscillation to the desired response. This way, it is possible to mimic different coupling coefficients, even with metal wires 13 of equal resistance. Such signals can be applied through wires (not shown) connecting electrical contacts to the outside world.

Alternatively, pairs of nearest-neighbor cavities may be connected by electrical conductors of distinct resistance, as described earlier. A further possibility is to rely on programmable resistive element 31, as discussed earlier too. In the latter case, the computer 101 may be operatively connected to resistive elements 31, for example, via a light source or electrical programming means, to control the programming of elements 31 in accordance with parameters of the problem to solve.

For completeness, system 1 may further include a liquid supply 106i and a liquid waste unit 106o, which are connected to the cavities, to allow reaction device 10, 10a to be replenished with the oscillating reaction liquid, if necessary. That is, liquid supply 106i and liquid waste unit 106o are connected to liquid inlet 21 and liquid outlet 22 of reaction device 10, 10a.

FIG. 7 shows a schematic overview of a preferred system architecture. In this example, the system relies on a single (master) computer 101, for example, a standard desktop computer using Ubuntu 20.04 as operating system and a standard kernel (for example, LINUX 5.4). A liquid flow rate controller 106 (connected to both liquid supply 106i and liquid waste unit 106o) and control unit 104 are connected to master computer 101 using Ethernet (via network switch 103). In variants, the device includes a capillary pump downstream of liquid waste unit 106o, or in a flow path between liquid supply 106i and liquid waste unit 106o, to ensure a desired flow rate. No controller 106 is required in that case.

Other communications (for example, to/from readout unit 107) can be ensured via a universal service bus (USB) hub 102. Signal generator 105 can be directly connected to and controlled by control unit 104. The standard kernel runs all required algorithms to control and communicate with components 104-107. All required software can for example be written in C++ 14 or python 3.

A final aspect of the present invention is now described in reference to FIG. 9. This aspect concerns a method of operating an oscillating reaction device 10, 10a, or system 1, such as described above. Essential features of this method have already been described, be it implicitly, in reference to the previous aspects of the invention. Therefore, such features are only briefly described in the following.

Device 10, 10a is provided at step S10. Structural elements 13, 31, 32 of the device may already map the problem the solve. For example, wires 13 may have been patterned to adequately adjust to the couplings as defined in the underlying Hamiltonian (more explanations below). In other implementations, device 10a may include programmable resistive elements 31, which are programmed at step S20 (either optically or electrically, this depending on actual elements 31), as assumed in the flow of FIG. 9.

Next, an oscillating reaction liquid is filled (step S30) into cavities 14, 15 of device 10, 10a, with a view to triggering an oscillating reaction. As explained earlier, the oscillating reaction liquid contains nonpolar molecules of a given molecular species. The oscillating reaction is subject to both a positive chemical coupling (excitatory coupling) and an inhibitory chemical coupling (antiphase oscillations) of the filled cavities.

The positive chemical coupling results from structural elements 13, 18, g. For example, where a flow path is defined between the cavities, filling S30 the oscillating reaction liquid into the cavities causes various species (including the nonpolar molecules) to diffuse between cavities, through flow path 17 filled by the oscillating reaction liquid. This contributes to the positive chemical coupling. Still, other types of structural elements, such as electrical connectors 13, 31, 32 may also contribute to the positive coupling, as explained earlier.

The inhibitory chemical coupling is mediated by the nonpolar molecules and is governed by the dimension of the portion of the matrix material 16 that separates first-neighbor cavities 14, 15. Once the cavities are filled with the oscillating reaction liquid, the nonpolar molecules of the oscillating reaction liquid diffuse through structured layer 16 from one cavity to the other, thereby yielding an inhibitory coupling.

Electrochemical or optical measurements are performed (step S60) from within the cavities. Such measurements can notably be performed with a view to solving a QUBO problem or an Ising problem, as assumed in FIG. 9.

In that case, the solution to the problem is achieved by measuring the phases in all relevant cavities. Plus, the reaction oscillations can be forced to a given frequency. In that case, the method further comprises applying S40-S50 signal pulses (either optical or electrical signals) to the cavities, in accordance with a desired oscillation frequency, to force (and thus lock) the oscillations across the cavities (that is, the oscillators) to the desired frequency.

However, whether to force the oscillations depends on the problem to solve. Forcing oscillations is convenient way to implement an Ising Hamiltonian as defined below. A non-forced system would amount to computing a more complex Hamiltonian, which can be of interest for certain applications.

In variants, the forcing is applied gradually, after the system has already evolved. This effectively amounts to implementing a “thermal annealing” method, which helps the system to overcome local minima, as further discussed below.

In addition, the system of cavities can possibly be reset by applying a string light pulse, to eliminate oscillations in all cavities. After applying such a pulse, the oscillations start again in all cavities, which effectively results in restarting the calculation. Doing this is useful to gather statistics and help find the optimal solution of complex problems.

When willing to solve an Ising Hamiltonian (see the equation below), the signal pulses can be applied by generating S40 clock signals and then applying S50 pulsed signals in accordance with generated clock signals. Use is preferably made of a computer 101, which control the readout unit and provides all the necessary parameters of the problem to solve, that is, the Ising parameters.

Several chemical oscillation cycles are typically needed to reach a solution, which may require replenishing S35 cavities with reaction liquid. The oscillation period depends on the concentrations used in the liquid and the external temperature. A typical example of electrochemical pulse sequence is the following. Given an oscillation period of 34 s, the electrochemical pulse sequence has a period of 17 s, corresponding to twice the frequency. Each sequence is decomposed into three subsequences, the respective durations of which are 6 s, 6 s, and 5 s. During the first subsequence, a first pulse (+1.2 V) is applied for 5 s, followed by a flat signal (1 s, 0 A). The second subsequence is similar, except that the voltage bias is now of −1.2 V. No signal is applied during the last subsequence. The whole sequence is repeatedly applied to ensure several cycles of forced oscillations, as necessary to reach a solution.

In more detail, the signal pulses are applied to synchronize oscillations with the external frequency. More precisely, the applied pulses make it possible to force a phase of the oscillating reaction. This phase is normally either equal to 0° or 180°, to reflect spins involved in the Ising Hamiltonian. Typically, the frequency used is close to two times the inherent chemical frequency of the reaction. Thus, the liquid volumes contained in the cavities can register in phase with the first or second oscillation of the clock, that is, either at 0° degrees or 180° phase shift with respect to the external clock.

The aim is to solve a Hamiltonian Hi, defined as:

Hᵢ=Σ_{i<j}Jᵢⱼsᵢsⱼ−Σᵢhᵢsᵢ.

This Hamiltonian describes coupled spins si, sj. The first term on the right-hand side describes the spin-spin interaction with coupling constant Jij, while the second term is the response to an external (magnetic) field h.

When the dimension of the problem is equal to or exceeds 2, the problem is known to be an NP-hard problem for classical computers. The problem is called an Ising problem if all hi are equal to zero, else the problem is a QUBO problem. In principle, the techniques disclosed herein make it possible to implement a chemical solver for Ising or QUBO problems. Such problems generally problems require connections between all neighbors of adjustable coupling strength Jij. The present approach provides this adjustable coupling strength. However, by construction, the coupling strength can only be fully adjusted between nearest neighbors. For problems that require a higher number of coupled cells, mapping algorithms are known, which can be used to map the problem onto a nearest-neighbor implementation.

The fundamental quantity in a Hamiltonian as defined above is the spin s. A spin can only have two possible states with respect to a measurement axis, whereas a chemical oscillator has a continuous phase. In order to enforce a two-state system for oscillators, an external clock of half the oscillation frequency is used. Thus, the oscillators have two possible phases to synchronize with the external clock, shifted by 180 degrees. A spin implementation can accordingly be realized. In the present case, the external clock can be imposed by light pulses or electrochemical signals, that is, voltages signals applied on a wire connected to contacts 12.

The energy landscape of the Ising Hamiltonian can be very complex, exhibiting many local minima, especially for large system sizes. Starting from a certain arrangement of the spins, the system may become stuck in a local minimum, which may be far from the optimal solution. For this reason, annealing methods can be used. Annealing means raising the “temperature” of the system, thereby enhancing internal noise, providing enough energy to overcome local minima. The “temperature” is then gradually reduced until the solver is forced into a certain state. Compared to spins, the continuous phase of the chemical oscillations is a much less constraint system. Thus, it can explore the energy landscape in a higher dimensional space, thereby avoiding local minima. The effect is similar to “thermal annealing”, which provides energy in the form of thermal noise to overcome local minima. The temperature is slowly decreased to force the system to settle into a (local or global) minima. The annealing process enhances the likelihood to find the global solution. Similarly, here, the external force is slowly increased, the system can explore the phase states until it settles to a final state.

While the present invention has been described with reference to a limited number of embodiments, variants and the accompanying drawings, it will be understood by those skilled in the art that various changes may be made, and equivalents may be substituted without departing from the scope of the present invention. In particular, a feature (device-like or method-like) recited in a given embodiment, variant or shown in a drawing may be combined with or replace another feature in another embodiment, variant or drawing, without departing from the scope of the present invention. Various combinations of the features described in respect of any of the above embodiments or variants may accordingly be contemplated, that remain within the scope of the appended claims. In addition, many minor modifications may be made to adapt a particular situation or material to the teachings of the present invention without departing from its scope. Therefore, it is intended that the present invention is not limited to the particular embodiments disclosed, but that the present invention will include all embodiments falling within the scope of the appended claims. In addition, many other variants than explicitly touched above can be contemplated. For example, components of device 10, 10a may be made of other materials. In particular, any matrix material 16 that is amenable to both processing cavities (at the nano or microscale) and permeation of small nonpolar molecules can be contemplated.

### Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

Including/include/includes: unless otherwise explicitly noted, means “including but not necessarily limited to.”

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Set of thing(s): does not include the null set; “set of thing(s)” means that there exist at least one of the thing, and possibly more; for example, a set of computer(s) means at least one computer and possibly more.

What is claimed is:

1. An oscillating reaction device comprising:
a structured layer of a matrix material; and
a plurality of structural elements;
wherein:
a plurality of cavities are defined in the structured layer, with the cavities having a size and shape such that the cavities can be filled with an oscillating reaction liquid that includes nonpolar molecules of a given molecular species to trigger an oscillating reaction; and
respective central axes of the cavities are arranged according to a 2D lattice sized and shaped such that each pair of nearest-neighbor cavities of the plurality of cavities is separated by a portion of the matrix material;
the portions of matrix material separating each pair of cavities is selectively permeable to the nonpolar molecules, to yield an inhibitory chemical coupling of filled cavities of each pair of nearest neighbor cavities so that the inhibitory chemical coupling is mediated by the nonpolar molecules in operation; and
the structural elements of the plurality of structural elements connect the cavities to yield a positive chemical coupling of the filled cavities, in operation.

2. The oscillating reaction device according to claim 1, wherein:
the device further includes electrical contacts defined to allow electrochemical measurements to be performed from within the cavities, in operation.

3. The oscillating reaction device according to claim 1, wherein:
the device is further configured to allow pulsed signals to be applied to the cavities to force the oscillating reaction in accordance with an external clock, in operation.

4. The oscillating reaction device according to claim 1, wherein:
the cavities are defined as blind holes in the structured layer, whereby the cavities are open on a surface of the structured layer, on one side thereof, and
the cavities of each pair of the pairs of nearest-neighbor cavities are laterally separated by the portion of matrix material, the portion extending along a direction that is parallel to the surface, over a distance that is less than or equal to an average diffusion distance of the nonpolar molecules through the matrix material over a period of oscillating reaction, whereby nonpolar molecules of the given molecular species can diffuse through the structured layer from one cavity to the other cavity of the each pair to yield the inhibitory coupling, in operation.

5. The oscillating reaction device according to claim 4, wherein the distance is between 1 and 10 μm.

6. The oscillating reaction device according to claim 4, wherein the plurality of structural elements are respectively structured to define a flow path between the cavities, whereby the flow path can be filled by the oscillating reaction liquid to form a diffusion flow path, through which the nonpolar molecules can diffuse from one of the cavities to another of the cavities to at least contribute to the positive chemical coupling.

7. The oscillating reaction device according to claim 6, wherein the structural elements are structured to define one of a gap and a trench, thereby defining the flow path.

8. The oscillating reaction device according to claim 7, wherein the structural elements further include a lid, which extends over the surface on the one side, at a distance of the surface, to define the gap between the lid and the surface.

9. The oscillating reaction device according to claim 8, wherein the gap is between 0.001 and 1 μm.

10. The oscillating reaction device according to claim 6, wherein the structural elements further include electrical connectors, each connecting interiors of the cavities of a respective one of the pairs of nearest-neighbor cavities.

11. The oscillating reaction device according to claim 10, wherein:
the device further includes one or more basis layers, which extends parallel to the structured layer on another side thereof, opposite to the one side; and
the electrical connectors are at least partly arranged in and/or on one or more of the basis layers.

12. The oscillating reaction device according to claim 11, wherein:
the electrical connectors include noble metal wires that are patterned in or on one of the basis layers, so as for each of the noble metal wires to be exposed to interiors of each of the cavities of the respective one of the pairs; and
at least some of the noble metal wires have distinct electrical resistances.

13. The oscillating reaction device according to claim 10, wherein the electrical connectors include, each, a programmable resistive element.

14. The oscillating reaction device according to claim 1, wherein the matrix material is a nonpolar material, which includes a polymer that is one of polydimethylsiloxane and SU-8.

15. A method of operating an oscillating reaction device, wherein the method comprises:
providing a reaction device comprising a structured layer of a matrix material, in which cavities are defined and centers of the cavities are arranged according to a 2D lattice, whereby pairs of nearest-neighbor cavities of the cavities are, each, separated by a portion of the matrix material, the portion being selectively permeable to nonpolar molecules of a given molecular species and structural elements connecting the cavities;
filling an oscillating reaction liquid into the cavities, the oscillating reaction liquid containing nonpolar molecules of the given molecular species, to trigger an oscillating reaction that is subject to a positive chemical coupling of filled cavities of the pairs, the positive chemical coupling resulting from the structural elements and an inhibitory chemical coupling of the filled cavities of the pairs, the inhibitory chemical coupling mediated by the nonpolar molecules and governed by a dimension of the portion of the matrix material separating the cavities of each of the pairs; and
performing electrochemical or optical measurements from within the cavities.

16. The method according to claim 15, further comprising:
applying signal pulses to the cavities according to an oscillation frequency to force the oscillating reaction across the cavities at the oscillation frequency.

17. A device comprising:
a structured layer of a matrix material;
a plurality of structural elements; and
an oscillating reaction liquid that includes nonpolar molecules of a given molecular species suitable for being subject to an oscillating reaction;
wherein:
a plurality of cavities are defined in the structured layer;
the cavities of the plurality of cavities are filled with the oscillating reaction fluid;
respective central axes of the cavities are arranged according to a two dimensional lattice sized and shaped such that each pair of nearest-neighbor cavities of the plurality of cavities is separated by a portion of the matrix material;
the portions of matrix material separating each pair of cavities is selectively permeable to the nonpolar molecules, to yield an inhibitory chemical coupling of filled cavities of each pair of nearest neighbor cavities so that the inhibitory chemical coupling is mediated by the nonpolar molecules during the oscillating reaction; and
the structural elements of the plurality of structural elements connect the cavities to yield a positive chemical coupling of the filled cavities during the oscillating reaction.

18. The device of claim 17 further comprising:
a pulsing device structured and located to apply signal pulses to the cavities according to an oscillation frequency to force the oscillating reaction across the cavities at the oscillation frequency.

19. The device of claim 18 further comprising:
an electrochemical measuring device, structured, located and connected to perform electromechanical measurements from within the cavities.

20. The device of claim 18 further comprising:
an optical measuring device, structured, located and connected to perform optical measurements from within the cavities.
